(19) United States
(12) Patent Application Publication
(10) Pub. No.: US 2011/0105900 A1
(43) Pub. Date: May 5, 2011

(54) Transducer Unit Incorporating an Acoustic Coupler

(75) Inventor: Robert Randall Entrekin, Kirland, WA (US)

(73) Assignee: Koninklijke Philips Electronics N.V., Eindhoven (NL)

(21) Appl. No.: 11/570,171

(22) PCT Filed: Jun. 9, 2005
(86) PCT No.: PCT/IB2005/051900

§ 371 Date: Dec. 7, 2006

Related U.S. Application Data
(60) Provisional application No. 60/579,551, filed on Jun. 14, 2004.

(51) Int. Cl. A61B 8/14

(52) U.S. Cl. 600/443

(57) ABSTRACT

The present invention relates to a transducer unit for an ultrasonic breast imaging system, in which an acoustic coupler is employed which comprises a frame in which a transducer is mounted. The frame is open at one end, and a surface of the transducer is exposed at the open end. The frame and exposed transducer surface are put under compression against the surface of a compression plate with a gasket, or similar, providing a fluid-tight seal therebetween. A thin layer of acoustic couplant is provided between the transducer surface and the compression plate. The frame defines a chamber around the nonexposed surface of the transducer, the chamber housing an acoustic couplant.

### FIELD OF THE INVENTION

This invention relates to an acoustic coupler for a transducer unit for use in an ultrasonic imaging system and, more particularly, to a transducer unit which is especially suited for use in an ultrasonic breast system arranged and configured to acquire ultrasound scans of a compressed breast for use in breast evaluation and/or adjunctive ultrasound mammography, or other applications requiring reliable and repeatable three-dimensional breast ultrasound data.

### BACKGROUND OF THE INVENTION

Ultrasonic imaging has rapidly become the preferred modality for the non-invasive investigation of human tissues. In particular, ultrasonic imaging has been used for many years to produce diagnostic images of the breast. For example, U.S. Pat. No. 4,298,009 describes a breast scanning arrangement consisting of a patient table on which a patient lies in the face-down position. The patient table is provided with a hole through which the patient suspends a breast into a tank of water located below the table. At the bottom of the tank, there is provided an ultrasound transducer directed upward toward the breast. The transducer is rocked back and forth as it transmits a beam of ultrasound to the breast, thereby scanning the plane of the breast. The transducer is mounted on a mechanism, which can move the transducer perpendicular to the image plane (in the elevation direction), thereby enabling the scan plane to be positioned to scan another plane of the breast. The ultrasonic energy travels to and from the breast through the water in the tank and, since ultrasound travels through water at nearly the same speed (1540 m/s) as it propagates through body tissue, the water in the tank provides an efficient coupling of ultrasonic energy between the transducer and the breast tissue.

More recent attempts to automate breast ultrasound have used similar methods to mammography, where the breast is compressed between two semi-rigid surfaces and all such implementations provide acoustic coupling between the transducer and the compression plate, either by some form of water bag, by using a sponge around the transducer moistened by a coupling fluid, or by “pooling” coupling gel or liquid in the top of a standard mammographic compression plate. In these implementations, both the thickness and shape of the fluid gap between the transducer and the breast support plate is uncontrolled and variable over the scanning area. If the speed of sound of the coupling fluid does not closely match the average speed of body tissue, then significant refraction and defocusing of the ultrasound beam can result, causing image distortion and degradation of image quality. Thus, these implementations rely on using a coupling fluid with a speed of sound that is a close match to that of human body tissue (i.e. 1540 metres/second). This severely restricts the choices of fluids available for use, and complicates the design of such systems. Furthermore, the above-mentioned coupling methods are heavy, messy and subject to leakage, which factors restrict the use of such systems in a clinical environment. Most of them also rely on gravity to contain the liquid, and therefore restrict the compression plate to a horizontal orientation, thereby eliminating the option for lateral or oblique compression of the breast tissue.

### SUMMARY OF THE INVENTION

It is an object of the present invention to provide an acoustic coupler for a transducer unit of an ultrasonic imaging system which overcomes the problems outlined above, and allows for the use of coupling fluids with desirable chemical and mechanical properties, irrespective of their speed of sound, acoustic impedance, attenuation, or other acoustic properties.

In accordance with the present invention, there is provided an ultrasonic breast imaging system in which a breast to be imaged is placed under compression by at least one compression plate and ultrasonically scanned through said plate, said system comprising:
a) an ultrasonic transducer mounted in a frame having an open end defined by a peripheral edge, a surface of said transducer being exposed relative to said frame and substantially aligned with said peripheral edge;
b) means for placing said exposed surface of said transducer and said peripheral edge of said frame in compression against a surface of said compression plate with a layer of couplant fluid between said exposed surface of said transducer and said surface of said compression plate; and
c) means for providing a substantially fluid-tight seal between said peripheral edge of said frame and said surface of said compression plate.

Also in accordance with the present invention, there is provided a method of manufacturing an ultrasonic breast imaging system in which a breast to be imaged is placed under compression by at least one compression plate and ultrasonically scanned through said plate, said method comprising:
a) mounting an ultrasonic transducer in a frame having an open end defined by a peripheral edge, leaving a surface of said transducer exposed relative to said frame and substantially aligned with said peripheral edge;
b) placing said exposed surface of said transducer and said peripheral edge of said frame in compression against a surface of said compression plate with a layer of couplant fluid between said exposed surface of said transducer and said surface of said compression plate; and
c) providing a substantially fluid-tight seal between said peripheral edge of said frame and said surface of said compression plate.

The present invention also extends to a transducer unit for use in an ultrasonic breast imaging system as defined above, the transducer unit comprising an ultrasonic transducer mounted in a frame having, an open end defined by a peripheral edge, a surface, of said transducer being exposed relative to said frame and substantially aligned with said peripheral edge, and means for receiving sealing means for creating a substantially fluid-tight seal between said peripheral edge of said frame and a surface of a compression plate.

Thus, the ultrasonic breast imaging system and the transducer unit of the present invention employ an acoustic coupler which provides a compact, lightweight, leak proof and reliable way to couple the transducer, in an ultrasonic imaging system, to the compression plate, in any orientation.

In a preferred embodiment, the compression plate may comprise a membrane under tension.

Beneficially, the interior of the frame defines a chamber around the transducer. This chamber is preferably filled with an acoustic couplant fluid. Beneficially, there may be provided at least one channel extending from inside the chamber to the exterior thereof. In one preferred embodiment, an inlet and an outlet are provided extending between the inside of the chamber and the exterior thereof. Means, such as a pump, are beneficially provided for creating a flow of acoustic couplant fluid through the chamber between the inlet and the outlet. Control means may be provided for controlling the pressure of acoustic couplant fluid within the chamber. A reservoir may be provided, from which couplant fluid is supplied to the chamber and/or to which couplant fluid from the chamber is directed.

These and other aspects of the invention will be apparent from and will be elucidated with reference to the embodiments described hereinafter.

### BRIEF DESCRIPTION OF THE DRAWINGS

The present invention will now be described in more detail, by way of example, with reference to the accompanying drawings, wherein:

FIG. 1 is a schematic perspective view of a transducer unit according to an exemplary embodiment of the present invention;

FIG. 2 is a schematic cross-sectional view of a transducer unit according to an exemplary embodiment of the present invention;

FIG. 3 is a schematic illustration of the basic construction of an ultrasonic breast imaging system according to an exemplary embodiment of the present invention;

FIG. 4 is a schematic perspective view of a “Whole Breast Ultrasound” ultrasonic breast imaging system according to an exemplary embodiment of the present invention;

FIG. 5 is a schematic fluid circulation diagram illustrating the manner of fluid circulation through a transducer unit according to an exemplary embodiment of the present invention.

### DETAILED DESCRIPTION OF THE INVENTION

Thus, as mentioned above, ultrasonic imaging has been used for many years to produce diagnostic images of the breast. Ultrasound systems are now being developed that, unlike earlier systems, produce a volumetric (3D) image of the breast. This may be done by scanning the breast with a moving array transducer. An array transducer can transmit and receive electronically steered beams, which can scan a plane of a subject without moving the transducer. As the array transducer is moved in the elevation direction, it successively scans a sequence of planes. The planes can be considered to be analogous to a pack of playing cards which are aligned in a pile. The plane of each playing card is a planar image, and a stack of the cards comprises a stack of planar images of a volumetric object which are aligned in parallel. The data of such a sequence of planes can be sued to render three-dimensional (3D) images, as is known in the art.

In order to scan the, breast accurately with a moving transducer, it is necessary to immobilise the breast while it is being scanned. If the breast is not immobilised and were to move while it is being scanned, a distorted image would result. A good way of immobilising the breast is by means of the use of compression plates, as is done in mammography systems and, as explained above, more recent attempts to automate breast ultrasound have used methods similar to mammography, where the breast is compressed between two semi-rigid surfaces. In some cases, this has been for the purpose of performing breast ultrasound only, for example, as described in US Patent Publication No. 2003/0007598. More commonly, the compression has been for the purpose of combining ultrasound with X-ray mammography. A system which immobilises the breast for scanning with compression plates can thus be designed for both mammographic and ultrasonic imaging on the same instrument, as described in, for example, U.S. Pat. No. 5,938,613.

When the breast is immobilised for scanning, it must be decided how to scan the breast. A preferred way is to ultrasonically scan the breast through one of the plates, as illustrated in the arrangement described in U.S. Pat. No. 6,574,499. However, in order to do this, the ultrasonic energy must be coupled into and out of the plate, and then into the breast, all with materials that closely match the speed of sound through the body, as discussed above. One way to do this is described in U.S. Pat. No. 6,574,499, whereby the upper plate comprises a plastic dish which contains a small amount of couplant (oil, in this case). The ultrasound transducer scans through the dish, using the couplant in the dish as the acoustic couplant for the ultrasonic energy. As the plastic dish compresses the breast, it is caused to bow upwardly in the centre due to pressure exerted by the compressed breast. The sides of the dish do not bend because they are made rigid by the vertical sides of the dish. The ultrasound transducer is then moved over the surface of the dish to scan the breast, with the oil in the dish providing the acoustic couplant. However, with the dish bowed upwards at its centre, a rigid translation device cannot be used to move the transducer and the solution proposed in U.S. Pat. No. 6,574,499 is to spring mount the probe vertically so that it can move up and down with the bowing of deformation of the centre of the dish.

However, there are several problems associated with this solution. Firstly, because the probe moves up and down with the deformed bottom of the dish, the resultant 3D image will be distorted due to the resultant motion. Secondly, the compression facility provided can only be used to perform vertical compression of the breast. If the tray were moved into any other position (to enable any other type of compression of the breast), the oil would pour out of the dish and damage the scanner. Thirdly, the variations in thickness of the oil-filled space between the transducer and the surface of the dish can distort and degrade the focus of the ultrasound beam emitted by the transducer. In order to minimise this, it is important to use oil or other couplant which is closely matched to the acoustic characteristics of the body being evaluated.

It is an object of the invention to enable the coupling of the transducer to the compression plate and to be able to move the transducer without distorting the image. It is another object of the invention to be able to choose a couplant for characteristics other than its acoustic speed of sound or acoustic impedance. It is a further object of the invention to be able to reposition the compression plates to different orientations, including vertically orienting the plates, without any loss of couplant.

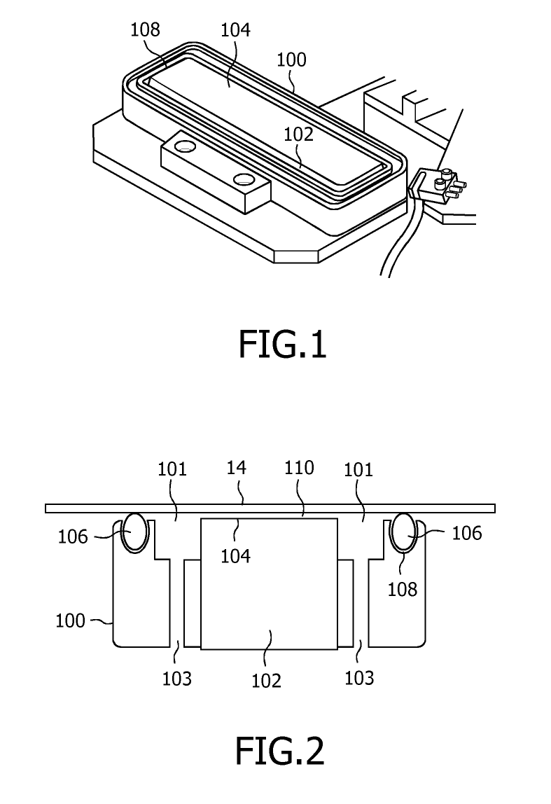

Referring to FIGS. 1 and 2 of the drawings, the present invention solves the above-mentioned problems associated with prior art arrangements by employing a small frame which forms a chamber 101 around the circumference of an array transducer 102, which frame 100 will hereinafter be referred to as a “puck”. The transducer array 102 is affixed inside the puck 100 with its exposed flat surfaced lens 104 aligned with, i.e. substantially in the same plane as, the upper surface of the puck 100. A pliant gasket 106 is provided in a peripheral groove 108 provided in the upper surface of the puck 100, which gasket 106 extends slightly upwardly beyond the plane of the upper surface of the puck 100 and the exposed transducer array lens 104 when the transducer unit is not mounted relative to a compression plate in an ultrasound breast scanner.

The transducer 102, puck 100 and gasket 106 are then placed in compression against the surface of a compression plate 14. A very small space 110 remains between the upper periphery of the exposed lens 104 of the array transducer 102 and the compression plate 14. In use, the chamber 101 and space 110 are filled with a small amount of fluid couplant via inlet and outlet channels 103.

The compressive force will form a dynamic sliding seal against the lower surface of the plate 14 which will retain the couplant inside the gasket 106. The compression of the lens 104 of the transducer against the plate 14 will cause only a thin, uniform thickness film of the couplant to remain between the transducer lens 104 and the plate 14. This thin film enables transmission of sound waves from the transducer lens 104 to the compression plate 14. The film is so thin as to have a negligible effect on the transmission of ultrasound, making the speed of ultrasound of the coupling fluid substantially irrelevant. Thus, the couplant can be chosen for other desirable properties, such as its lubricity, its chemical stability, its compatibility with the other materials used in the scanner, and its lack of toxicity with human tissue.

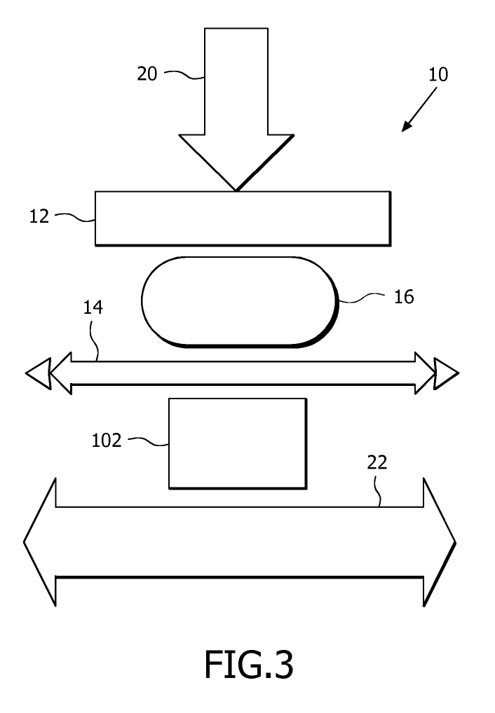

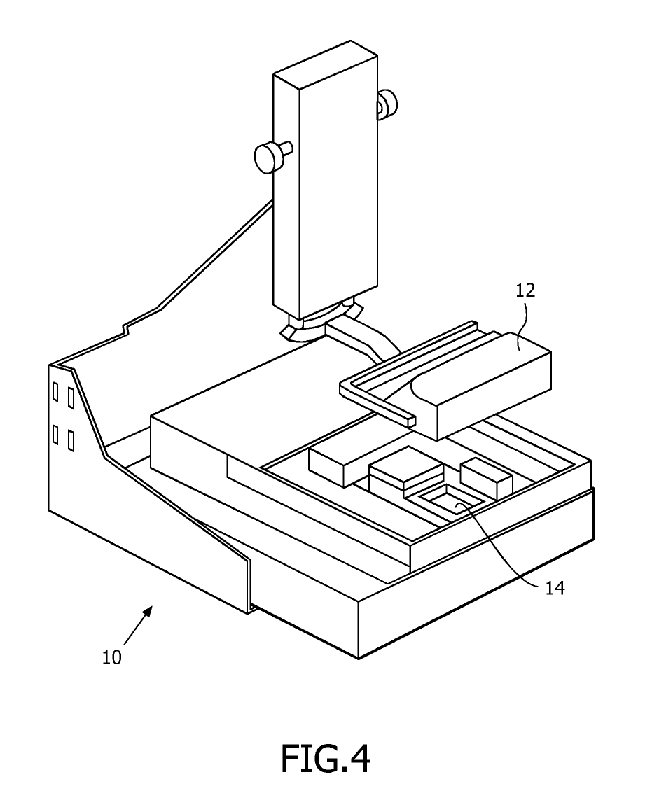

Referring to FIGS. 3 and 4 of the drawings, an exemplary breast scanning system 10 comprises upper and lower compression plates 12, 14, and the breast 16 to be scanned is retained between these two compression plates 12, 14. In the illustrated embodiment, the lower compression plate 14 is fixed in location and the upper compression plate is moveable to apply a downward compression force which retains the breast 16, as indicated by the compression force arrow 20. The compressed breast 16 is scanned by an ultrasound transducer 102 located below the lower compression plate 14. The transducer 102 scans the breast by articulation of the transducer in two dimensions by a mechanical motion system 22. In one embodiment, the transducer 102 may be driven by a motor along a rail in either of two opposite directions in one dimension. The rail is motor driven along two other rails which provides motion in the opposing directions of a second, orthogonal dimension. One dimension would be into and out of the drawing plane, for example.

It will be appreciated that the entire system 10 of FIG. 3 could also be constructed in an inverted configuration, i.e. the ultrasound transducer 102 could scan the breast 16 from above an upper compression plate and either of the compression plates could move to apply the compressive force.

In this exemplary embodiment of the present invention, the compression plate 14, against the surface of which the transducer 102, puck 100 and gasket 106 are placed in compression, may comprise a thin membrane which is under tension; as described in U.S. Pat. No. 6,682,484 in which case the compressive force referred to above can be created by placing the upper surface of the puck 100 slightly (a few millimeters) above the plane of the membrane (somewhat like a tent pole holding up a tent). The lateral tension in the membrane creates a downward compressive force component against the slightly elevated puck, which seals the gasket. In the case of a rigid compression plate instead of a tensioned membrane, the compressive force to seal the gasket can be provided by springs or other compliant mounting means which force the puck against the compression plate.

In any event, and referring back to FIG. 2 of the drawings, the gasket 106 will be placed under compression by the resultant compressive force and will form a dynamic seal against the surface of the paddle 14 which will retain the couplant inside the gasket 106. The compression of the lens 104 of the transducer against the paddle 14 will cause only a thin coating of the couplant to remain between the transducer lens 104 and the paddle 14. As already mentioned above, this thin coating will have a negligible effect on the acoustic coupling of ultrasound, making the speed of ultrasound through the coupling substantially irrelevant in the choice of couplant used. Thus, the couplant can be chosen for other desirable properties, such as its lubricity, its corrosiveness (or lack thereof), its lack of volatility, its compatibility with the other materials used in the scanner, and its compatibility with human tissue.

Preferred fluids include hydrocarbon mineral oils, such as Invoil 20 or Inland 45 (Inland Vacuum Industries, Churchville, N.Y. USA), silicone oils such as DC 702 or DC 704 (Dow Coming, Midland, Mich., USA), and perfluorinate oils such as Krytox 1506 or Krytox GPL 102 (Dupont, Wilmington, Del., USA). These oils generally have very low speeds of sound (700 to 1300 meters per second, which do not match the 1540 m/s speed of body tissue. However, they are desirable couplant fluids because they have excellent lubricity and compatibility with plastics, they are chemically stable, and they are non-toxic to humans. The present invention overcomes their acoustic limitations, and allows them to be used for their desirable mechanical and chemical properties. Nonetheless, it will be obvious to those skilled in the art that the present invention does not preclude the use of fluid couplants that more closely match the speed of 1540 meters per second, such as alcohols, glycols, glycerol, or aqueous solutions thereof, as well as pure water.

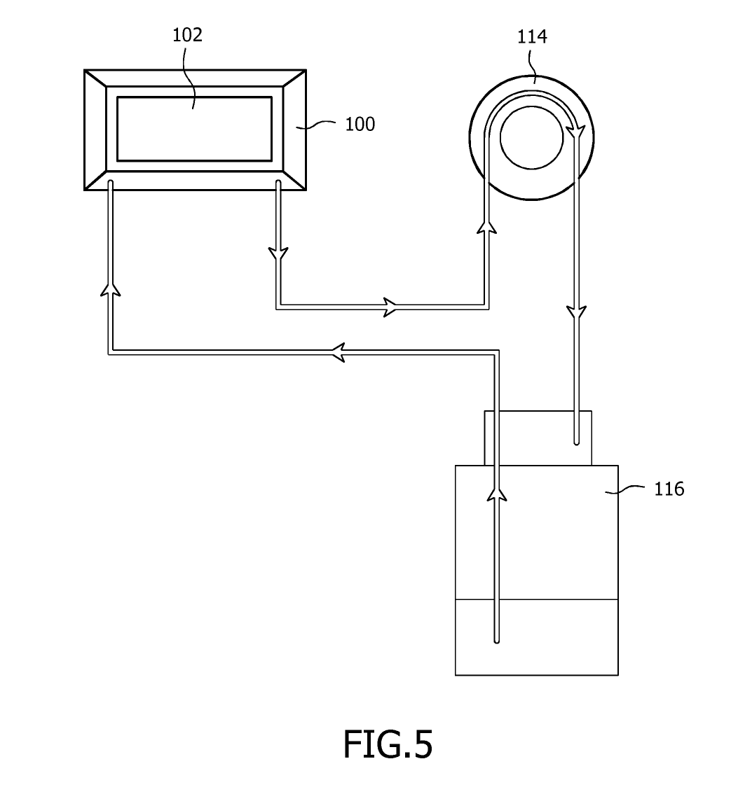

In a constructed embodiment, a steady flow of fluid couplant is supplied to the chamber 101 and space 110 defined by the puck 100 by a pump. Referring to FIG. 5 of the drawings, a roller pump 114 may be provided which can supply couplant from a reservoir bottle 116 to the space defined by the puck 100 surrounding the transducer 102. The pump 114 can also withdraw couplant from the puck 100 by negative pressure, which prevents couplant under pressure from being forced through the gasket seal into other parts of the scanner. Applying negative pressure to the puck also insures that any air which may leak past the gasket and into the chamber 101 will be sucked out and replaced by fluid from the reservoir 116. Air bubbles in the flow of couplant are kept away from the space 110 between the transducer lens 104 and the plate 14, first by the force of the lens 104 against the plate 14 which makes the space 110 small, second by the surface tension of the bubble which prevents it from entering into the small space 110, and third by the flow of couplant through the chamber 101 surrounding the transducer 102, which continually flushes bubbles out of the puck 100 and returns them to the reservoir where they cannot be recirculated, since the fluid is drawn from the bottom of the reservoir bottle.

It will be appreciated that, in a constructed embodiment, the transducer and membrane could be rotated from an orientation with the transducer below the plate to an orientation with the transducer above the plate, without any fluid leaks of the couplant.

Thus, the exemplary embodiment of the invention described above provides a method and apparatus for providing continuous acoustic coupling between a moving ultrasound transducer and a planar membrane under tension. The proposed exemplary embodiment provides a sliding seal between the transducer and the membrane, which prevents fluid leakage and continuously removes any air bubbles that may be entrained during the translation of the transducer, regardless of the orientation of the scanning apparatus.

It should be noted that the above-mentioned embodiment illustrates rather than limits the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. An ultrasonic breast imaging system in which a breast to be imaged is placed under compression by at least one compression plate and ultrasonically scanned through said plate, said system comprising:
a) an ultrasonic transducer mounted in a frame having an open end defined by a peripheral edge, a surface of said transducer being exposed relative to said frame and substantially aligned with said peripheral edge;
b) means for placing said exposed surface of said transducer and said peripheral edge of said frame in compression against a surface of said compression plate with a layer of couplant fluid between said exposed surface of said transducer and said surface of said compression plate; and
c) means for providing a substantially fluid-tight seal between said peripheral edge of said frame and said surface of said compression plate.

2. A system according to claim 1, wherein the compression plate comprises a membrane under tension.

3. A system according to claim 1, wherein the interior of the frame defines a chamber around the transducer.

4. A system according to claim 3, wherein the chamber is filled with an acoustic couplant fluid.

5. A system according to claim 3, comprising at least one channel extending from inside the chamber to the exterior thereof.

6. A system according to claim 5, wherein an inlet and an outlet are provided, extending between the inside of the chamber and the exterior thereof.

7. A system according to claim 6, comprising means for creating a flow of acoustic couplant fluid through the chamber between the inlet and the outlet.

8. A system according to claim 4, comprising control means for controlling the pressure of acoustic couplant fluid within the chamber.

9. A system according to claim 4, comprising a reservoir from which couplant fluid is supplied to the chamber and/or to which couplant fluid from the chamber is directed.

10. A method of manufacturing an ultrasonic breast imaging system in which a breast to be imaged is placed under compression by at least one compression plate and ultrasonically scanned through said plate, said method comprising:
a) mounting an ultrasonic transducer in a frame having an open end defined by a peripheral edge, leaving a surface of said transducer exposed relative to said frame and substantially aligned with said peripheral edge;
b) placing said exposed surface of said transducer and said peripheral edge of said frame in compression against a surface of said compression plate with a layer of couplant fluid between said exposed surface of said transducer and said surface of said compression plate; and
c) providing a substantially fluid-tight seal between said peripheral edge of said frame and said surface of said compression plate.

11. A transducer unit for use in an ultrasonic breast imaging system according to claim 1, the transducer unit comprising an ultrasonic transducer mounted in a frame having an open end defined by a peripheral edge, a surface of said transducer being exposed relative to said frame and substantially aligned with said peripheral edge, and means for receiving sealing means for creating a substantially fluid-tight seal between said peripheral edge of said frame and a surface of a compression plate.
